(19) United States
(12) Patent Application Publication
(10) Pub. No.: US 2020/0138522 A1
(43) Pub. Date: May 7, 2020

(54) 3D MODEL GENERATION USING THERMAL IMAGING AND X-RAY

(71) Applicant: Zimmer, Inc., Warsaw, IN (US)

(72) Inventor: Vinay Tikka, Winona Lake, IN (US)

(21) Appl. No.: 16/675,848

(22) Filed: Nov. 6, 2019

Related U.S. Application Data
(60) Provisional application No. 62/756,654, filed on Nov. 7, 2018.

(51) Int. Cl. A61B 34/10; A61B 5/01; A61B 6/00; A61B 5/00

(52) U.S. Cl. CPC A61B 34/10; A61B 5/015; A61B 2034/105; A61B 6/505; A61B 5/7264; A61B 6/5235

(57) ABSTRACT

A system and method for generating a 3D model of patient anatomy, including for example, a bone, soft tissue, or the like. A system may include a thermal image camera to generate a thermal image, an x-ray device to capture an x-ray image of a bone of a patient, and a processor coupled to memory. The processor may perform operations including generating a thermal map of the patient anatomy from the thermal image, and generate a 3D bone model of the bone from the x-ray image. A machine learning technique may be used to generate a 3D model of the patient anatomy including for example, the bone, soft tissue, or the like. The 3D model may be output for display.

### CLAIM OF PRIORITY

This application claims the benefit of priority to U.S. Provisional Application Nos. 62/756,654, filed Nov. 7, 2018, titled “3D MODEL GENERATION USING THERMAL IMAGINING AND X-RAY”; which is hereby incorporated herein by reference in its entirety.

### BACKGROUND

Producing a diagnostic image is part of the manufacturing process for Patient Specific Instrumentation (PSI). Diagnostic imaging may include a CT or an MRI. However, these modalities have an additional cost associated with them, in time, money, and potentially health. With the latest advancements in the statistical shape modeling, machine learning, and image processing algorithms, 2D images may be converted to 3D models. Specifically for orthopedic disorders and procedures, x-rays may be used to produce a 3D model. These models are an approximation of only bones, and thus fail to include any representation of muscle or soft tissue that surrounds the bones, due to the limitations of x-rays and other current imaging techniques.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a diagram showing components of a 3D model generation system in accordance with some embodiments.

FIGS. 2A-2D illustrates example thermal images of patient anatomy in accordance with some embodiments.

FIGS. 3A-3B illustrate a flowchart illustrating a technique for creating a patient model and accompanying depictions of aspects of the flowchart in accordance with some embodiments.

FIG. 4 illustrates a machine learning engine for determining feedback in accordance with some embodiments.

FIG. 5 illustrates a classifier machine learning training block diagram in accordance with some embodiments.

FIG. 6 illustrates a flowchart showing a technique for generating a 3D model using thermal imaging and an x-ray in accordance with some embodiments.

FIG. 7 illustrates a block diagram of an example machine upon which any one or more of the techniques discussed herein may perform in accordance with some embodiments.

### DETAILED DESCRIPTION

Systems and methods for creating a patient model, for example including a representation of bone and soft tissue, are described herein. The systems and methods herein may use an x-ray 3D bone model and a surface model or thermal map of soft tissue based on a thermal image to generate a 3D model of patient anatomy including bone and soft tissue. The 3D model may be used for reference in a surgical procedure, to produce Patient Specific Instrumentation (PSI), to produce a patient-specific implant or trial, or the like.

The 3D model may be generated from the bone model and the thermal map by using machine learning (e.g., deep learning) techniques, such as a convolutional neural network (CNN), a recurrent neural network (RNN), or the like. A machine learning technique may be trained using labeled data with a bone model or x-ray and a thermal map as inputs, labeled as corresponding to an output of a 3D model of patient anatomy including bone and soft tissue. In an example, a CNN or RNN may be trained by labeling a 2D x-ray with a thermal map generated from thermal images. The labeling may include identifying individual regions of the 2D x-ray and the thermal map where bone and soft tissue meet or are connected. In an example, a flattened surface model may be laid over the 2D x-ray, and then extruded to cover the 2D x-ray. The CNN may identify pixels, regions, or surfaces in a layer.

The 3D model of patient anatomy includes representations of both bone and soft tissue, including for example, a representation of ligaments and connection points to bone. The 3D model may include a representation of a knee, ankle, wrist, elbow, or the like. For example, the 3D model may include any joint where a 360 degree thermal image is possible. Other anatomy, such as shoulder, hip, etc., may be modeled with this technique, such as with a partial thermal map based on thermal imaging (e.g., less than a 360 degree view), with a resulting 3D model that partially maps the bone model to soft tissue (e.g., parts of soft tissue may be left out of the 3D model, where not captured in the thermal imaging). In an example, thermal imaging may be used to identify cartilage in a patient. In an example, thermal imaging may be used to identify a difference between diseased and normal tissue.

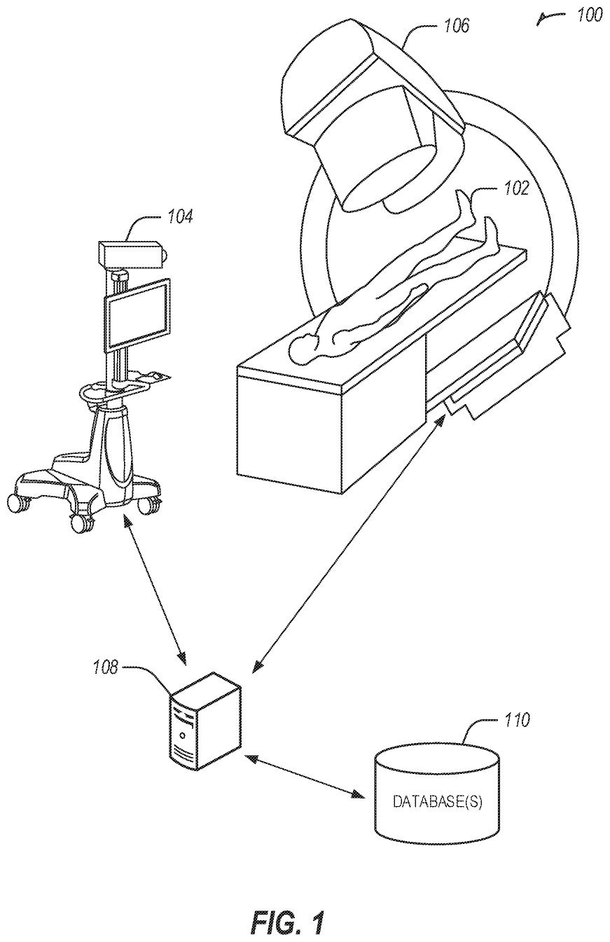

FIG. 1 illustrates a diagram showing components of a 3D model generation system 100 in accordance with some embodiments. The system 100 uses a thermal imager 104 to obtain thermal images from different views around a patient 102. The system 100 uses a diagnostic imaging device (e.g., an x-ray) to generate an image of a bone of the patient 102. The image of the bone and the thermal images may sent to a computing device 108 (e.g., a computer, a mobile device, a server, etc.) for processing. The computing device 108 may include a processor for combining the thermal images and the image of the bone as described herein. Images or results may be saved to a database 110, for example to train or update a machine learning technique based on the images or results.

In an example, the thermal imager 104 may be a thermal imaging camera such as a module attached to a mobile device, (for example, the FLIR ONE Pro from FLIR Systems of Wilsonville, Oreg.), or a stand-alone camera device (e.g., the FLIR E60 from FLIR Systems), which may be affixed to a cart, which may be moveable, wall, or other fixture or moveable apparatus, or may be hand-held. The thermal imager 104 may capture a plurality of images, such as two or more to capture depth, three or more for a 360 degree view, or four or more to capture anterior, posterior, medial and lateral views. Thermal images captured by the thermal imager 104 may be processed together to create a surface view of patient anatomy. For example, the computing device 108 may use an image stitching algorithm to stitch images together to create a panoramic surface view of anatomy of the patient 102. In an example, a single thermal imager may be used while being rotated around a joint or a portion of a joint to capture a panoramic thermal image (e.g., surface image) of the joint or portion of the joint. In another example, multiple thermal imagers may be used (e.g., at different angles) to capture two or more images of a joint or a portion of a joint and the images may then be stitched together.

The image of the bone may be used to create a 3D model of the bone. In an example, multiple 3D models may be created, such as, in a knee, 3D models may be created for a femur, tibia, and fibula from the diagnostic x-ray (e.g., using an x-ray to 3D model technique, such as the X-ray-based Patient Specific Instrument, X-PSI™ Knee System for creating 3D models from Zimmer Biomet of Warsaw, Ind.). In an example, two or more x-rays may be used to create the 3D model using other techniques. The 3D bone model created from the diagnostic image may not include information related to soft tissue, muscle, or other anatomy of the patient 102. However, the diagnostic image may include patient anatomy information, such as an outline of outer skin of the patient 102. Using the patient anatomy information from the diagnostic image, the 3D bone model and the surface view of patient anatomy generated from the thermal images may be combined to generate a final 3D model that includes both bone and patient anatomy such as soft tissue combined.

Combining the 3D bone model with the stitched surface of the patient's outer surface may generate a realistic 3D model of patient's bone plus an approximation of the soft tissue. Generating the combination may be achieved by using a deep learning algorithm, such as a Convoluted Neural Network (CNN), a Recurrent Neural Network (RNN), or the like. A deep learning algorithm (a subset of machine learning, techniques of which may also be used) uses different layers to determine points or locations in the outline from the diagnostic image as a marker to match with locations of the stitched panoramic view of the patient's skin. Each layer may find pixels, regions, or features to fit the stitched surface with the 3D model of the bone. This is described in more detail below.

When the matching is done successfully, the deep learning algorithm may output a good approximation of the patient's anatomy with soft tissue and bone. This information may be used by surgeons in the pre-op planning of the surgery for the patient, such as by displaying a 3D model including bone and patient anatomy information on a user interface, which may be interactive. In an example, some post-processing may be performed on the 3D model to finish or smooth the anatomy or bone, for example by adding texture, color, etc. The output 3D model may be a computer-aided design (CAD) file, a CT-MRI image-type file, or the like.

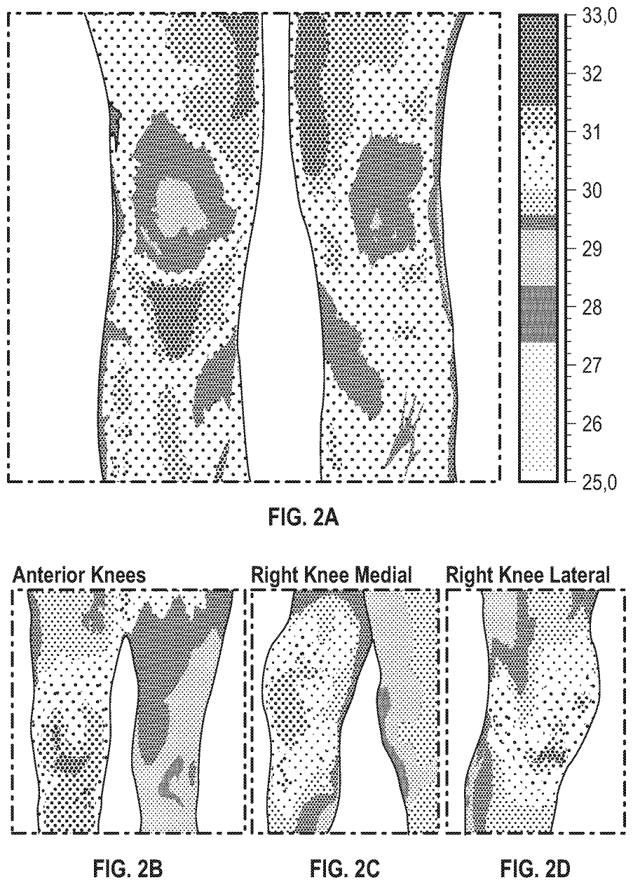

FIGS. 2A-2D illustrates example thermal images of patient anatomy in accordance with some embodiments. FIG. 2A illustrates a posterior view of a thermal image of a knee of a patient, FIG. 2B illustrates an anterior view of the knee, FIG. 2C illustrates a medial view of the knee, and FIG. 2D illustrates a lateral view of the knee. These four thermal images may be used to generate a thermal map of soft tissue of the knee.

In an example, the four views of the knee do not need to be perfectly aligned with each other or to a diagnostic image, but may be de-skewed, rotated, or otherwise adjusted in processing. Less than four of these views or different sets of views may be used (e.g., three images, each capturing 120 degrees to obtain a 360 degree view). In an example, previous thermal images may be used, such as to fill in gaps, establish general anatomy structures, or for scaling. For example, a thermal image from a patient of similar size may be used instead of a current patient image in one of the views depicted in FIGS. 2A-2D.

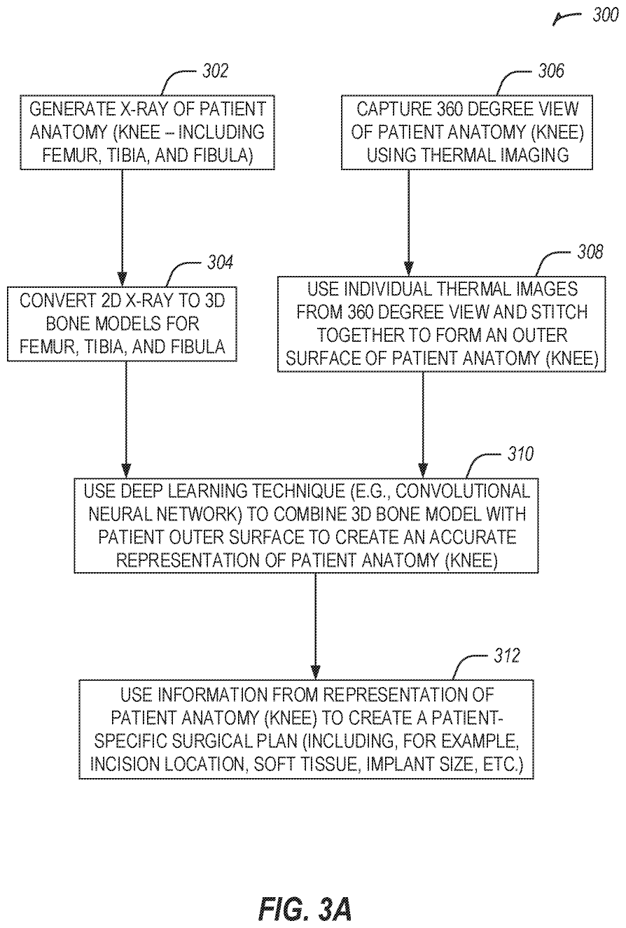

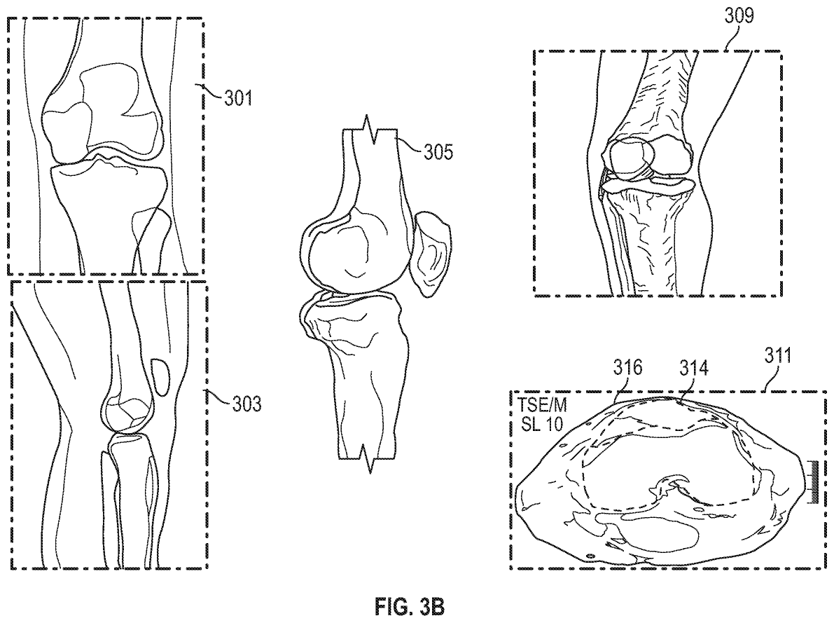

FIGS. 3A-3B illustrate a flowchart illustrating a technique 300 for creating a patient model and accompanying depictions of aspects of the flowchart in accordance with some embodiments.

The technique 300 includes an operation 302 to generate an x-ray of patient anatomy (e.g., including bone), for example, for a knee, of a femur, tibia, and fibula of the knee. Examples of x-rays of the patient anatomy are shown in FIG. 3B, for example x-ray 301 taken from a medial-lateral view and x-ray 303 taken from an anterior-posterior view.

The technique 300 includes an operation 304 to convert the 2D x-ray to a 3D bone model for each bone. For example, the 2D x-ray may be used with a second 2D x-ray to create a 3D bone model. The two x-rays may be combined using a system such as the X-Ray PSI System from Zimmer Biomet of Warsaw, Ind. The X-Ray PSI System involves the capture of two x-ray images of a patient knee anatomy from an x-ray source (e.g., a single x-ray source, an x-ray source configured to take two or more x-rays substantially simultaneously, or the like). The two x-ray images are subsequently combined in a computing system to create a three-dimensional image. To identify a common position from the two images, the patient wears one or more calibration objects which appear on the resultant x-ray images. From these images with the calibration objects, a 3D bone model may be generated. For example, a three-dimensional shape of the subject bone structure may be selected from an atlas, and this atlas shape is deformed and modified to match the characteristics of the three-dimensional space defined by the x-ray images. An example 3D bone model 305 generated from the x-ray 303 (or 301, or both) is shown in FIG. 3B.

The technique 300 includes an operation 306 to capture the patient anatomy using thermal imaging (e.g., a 360 degree view, such as with four or more thermal images). The patient anatomy may include soft tissue surrounding bone. Example thermal images are shown in FIGS. 2A-2D.

The technique 300 includes an operation 308 to use individual thermal images (e.g., four) to stitch an outer surface of the patient anatomy. For example, using the thermal images shown in FIGS. 2A-2D, an outer surface may be stitched. One of the images in FIGS. 2A-2D may be selected as a keyframe to start the stitching process. To stitch the next image onto the keyframe, standard features between two selected images are determined. When no common features between the keyframe image and selected image are found, then the technique 300 may continue to a next image to use as a keyframe. Once a keyframe image is selected with another image that has corresponding features, the technique 300 may stitch the two images together based on the features. This process may continue until all the images are stitched together. In an example, deep learning techniques (e.g., a deep neural network (DNN), such as a CNN or RNN) may be applied when the technique 300 fails to stitch an image automatically.

The technique 300 includes an operation 310 to use a deep learning technique (e.g., CNN or RNN) to combine the 3D bone model with the patient outer surface to generate an accurate representation of patient anatomy (including bone and soft tissue). The deep learning technique may use labeled data of a 3D model constructed (e.g., manually by a surgeon) from a 3D bone model, x-ray, and thermal images.

Using a DNN, the information from the x-ray, 3D bone model, and the thermal images (e.g., as the surface model) may be combined to create a 3D presentation of the patient's anatomy. A 3D image 311 shown in FIG. 3B illustrates an approximation of the final product from an axial view. The red outline 316 shows the images obtained from the thermal images and the green outline 314 represents the 3D bone model that was generated from the x-ray. In a DNN different layers may be used to find different features to match. For example, skin/muscle outline features of the x-ray (e.g., 301 or 303) may be matched with the stitched thermal images using a DNN. In an example, features from a 2D bone outline in the x-ray (e.g., 301 or 303) may be matched to features from the 3D bone model using a DNN. An example of a resulting 3D model 309 of the patient anatomy is shown in FIG. 3B.

The technique 300 includes an operation 312 to use information from the representation of the patient anatomy to create a patient-specific surgical plan, including, for example, an incision location, soft tissue placement or actions, an implant size or location, or the like.

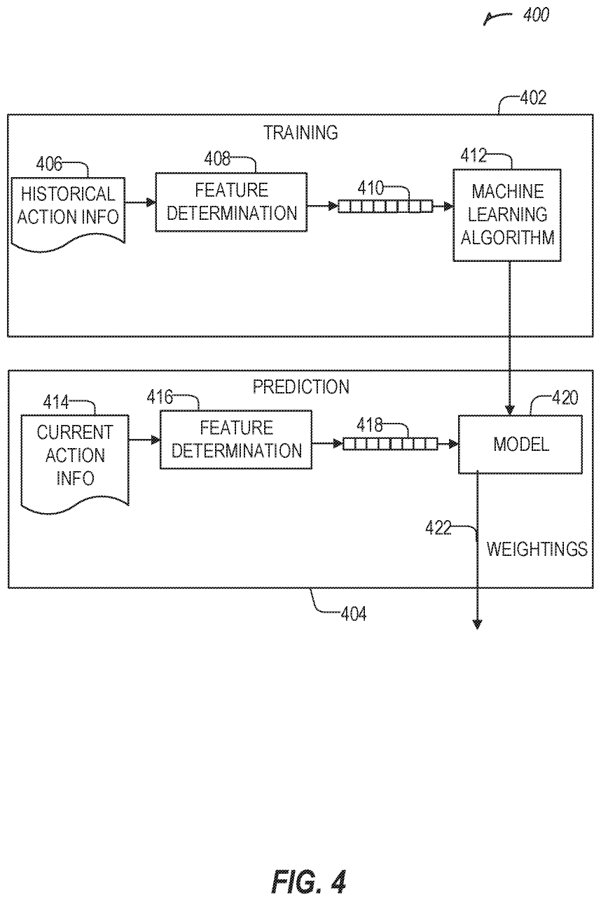

FIG. 4 illustrates a machine learning engine for determining feedback in accordance with some embodiments. A system may calculate one or more weightings for criteria based upon one or more machine learning algorithms. FIG. 4 shows an example machine learning engine 400 according to some examples of the present disclosure.

Machine learning engine 400 utilizes a training engine 402 and a prediction engine 404. Training engine 402 inputs labeled information 406 into feature determination engine 408. The labeled information 406 may include an x-ray, a 3D bone model, a thermal image, or a thermal map generated from a thermal image. The labeled information 406 may be labeled with a 3D model of patient anatomy including bone and soft tissue information.

Feature determination engine 408 determines one or more features 410 from this labeled information 406. Stated generally, features 410 are a set of the information input and include information determined to be predictive of a particular outcome. The machine learning algorithm 412 produces a model 420 based upon the features 410 and the labels.

In the prediction engine 404, current information 414 (e.g., an x-ray, 2D bone model, thermal image, or thermal map generated from a thermal image) may be input to the feature determination engine 416. Feature determination engine 416 may determine the same set of features or a different set of features from the current information 414 as feature determination engine 408 determined from the labeled information 406. In some examples, feature determination engine 416 and 408 are the same engine. Feature determination engine 416 produces feature vector 418, which is input into the model 420 to generate one or more criteria weightings 422. The training engine 402 may operate in an offline manner to train the model 420. The prediction engine 404, however, may be designed to operate in an online manner. It should be noted that the model 420 may be periodically updated via additional training.

The machine learning algorithm 412 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. Unsupervised models may not have a training engine 402. In an example embodiment, a neural network model is used and the model 420 is a CNN or an RNN.

In some examples a partial feature reconstruction may be generated, for example identifying a particular feature (e.g., where a ligament attaches to a bone), with other features generated using different models. In this example, multiple models may be used to identify one or more features.

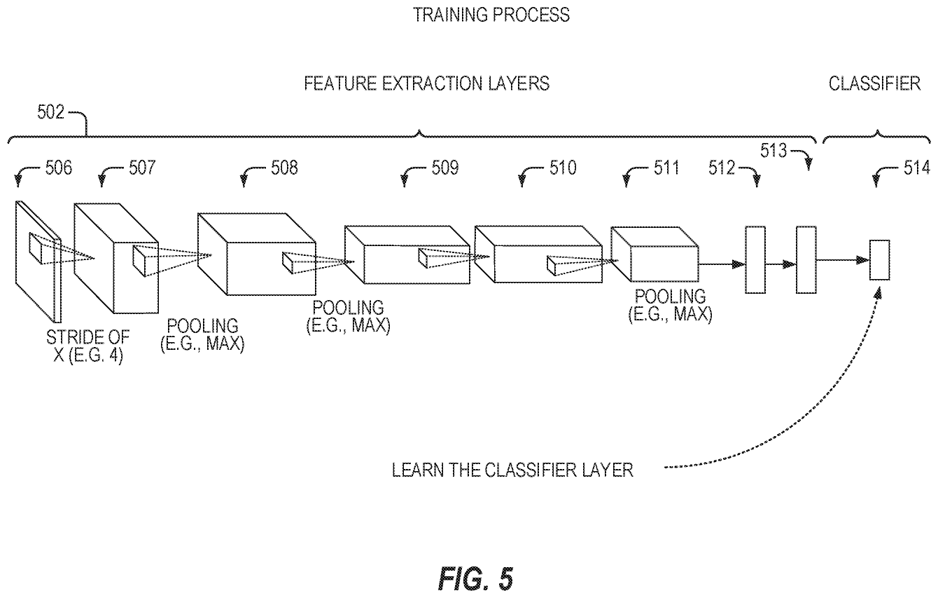

FIG. 5 illustrates a classifier machine learning training block diagram in accordance with some embodiments.

A neural network, sometimes referred to as an artificial neural network, is a computing system based on consideration of biological neural networks of animal brains. Such systems progressively improve performance, which is referred to as learning, to perform tasks, typically without task-specific programming. For example, in image recognition (e.g., recognizing an outline portion or location of an x-ray or soft tissue to bone attachment locations of patient anatomy in a thermal image), a neural network may be taught to identify locations that contain an object by analyzing example images that have a location tagged and, having learnt the location and tag, may use the analytic results to identify the location in untagged images. A neural network is based on a collection of connected units called neurons, where each connection, called a synapse, between neurons may transmit a unidirectional signal (e.g., in a CNN) or a feedback signal (e.g., in a RNN) with an activating strength that varies with the strength of the connection. The receiving neuron may activate and propagate a signal to downstream neurons connected to it, typically based on whether the combined incoming signals, which are from potentially many transmitting neurons, are of sufficient strength, where strength is a parameter (and in RNNs, a feedback component may influence whether to propagate the signal).

A deep neural network (DNN) is a stacked neural network, which is composed of multiple layers. The layers are composed of nodes, which are locations where computation occurs, loosely patterned on a neuron in the human brain, which fires when it encounters sufficient stimuli. A node combines input from the data with a set of coefficients, or weights, that either amplify or dampen that input, which assigns significance to inputs for the task the algorithm is trying to learn. These input-weight products are summed, and the sum is passed through what is called a node's activation function, to determine whether and to what extent that signal progresses further through the network to affect the ultimate outcome. A DNN uses a cascade of many layers of non-linear processing units for feature extraction and transformation. Each successive layer uses the output from the previous layer as input. Higher-level features are derived from lower-level features to form a hierarchical representation. The layers following the input layer may be convolution layers that produce feature maps that are filtering results of the inputs and are used by the next convolution layer.

In training of a DNN architecture, a regression, which is structured as a set of statistical processes for estimating the relationships among variables, may include a minimization of a cost function. The cost function may be implemented as a function to return a number representing how well the neural network performed in mapping training examples to correct output. In training, if the cost function value is not within a pre-determined range, based on the known training images, backpropagation is used, where backpropagation is a common method of training artificial neural networks that are used with an optimization method such as a stochastic gradient descent (SGD) method.

Use of backpropagation may include propagation and weight update. When an input is presented to the neural network, it is propagated forward through the neural network, layer by layer, until it reaches the output layer. The output of the neural network is then compared to the desired output, using the cost function, and an error value is calculated for each of the nodes in the output layer. The error values are propagated backwards, starting from the output, until each node has an associated error value which roughly represents its contribution to the original output. Backpropagation may use these error values to calculate the gradient of the cost function with respect to the weights in the neural network. The calculated gradient is fed to the selected optimization method to update the weights to attempt to minimize the cost function.

FIG. 5 illustrates the feature-extraction process and classifier training, according to some example embodiments. Training the classifier may be divided into feature extraction layers 502 and classifier layer 514. Each image is analyzed in sequence by a plurality of layers 506-513 in the feature-extraction layers 502.

In an example, determining relevant locations (e.g., connection locations of bone to soft tissue in an x-ray and thermal image or skin outline in an x-ray to skin in a thermal image) may use a similarity comparison between images in the gallery set and the query set, which may include a K-nearest-neighborhood (KNN) method to estimate the locations. In the ideal case, there is a good feature extractor (e.g., inter-class distance is always larger than the intra-class distance), and the KNN method is adequate to estimate the locations.

Feature extraction is a process to reduce the amount of resources required to describe a large set of data. When performing analysis of complex data, one of the major problems stems from the number of variables involved. Analysis with a large number of variables generally requires a large amount of memory and computational power, and it may cause a classification algorithm to overfit to training samples and generalize poorly to new samples. Feature extraction is a general term describing methods of constructing combinations of variables to get around these large data-set problems while still describing the data with sufficient accuracy for the desired purpose.

In some example embodiments, feature extraction starts from an initial set of measured data and builds derived values (features) intended to be informative and non-redundant, facilitating the subsequent learning and generalization steps. Further, feature extraction is related to dimensionality reduction, such as be reducing large vectors (sometimes with very sparse data) to smaller vectors capturing the same, or similar, amount of information.

Determining a subset of the initial features is called feature selection. The selected features are expected to contain the relevant information from the input data, so that the desired task may be performed by using this reduced representation instead of the complete initial data. DNN utilizes a stack of layers, where each layer performs a function. For example, the layer could be a convolution, a non-linear transform, the calculation of an average, etc. Eventually this DNN produces outputs by classifier 514. In FIG. 5, the data travels from left to right and the features are extracted. The goal of training the neural network is to find the parameters of all the layers that make them adequate for the desired task (e.g., generating a 3D model that includes a bone element and a soft tissue element based on input of an x-ray, a 3D bone model, and a surface map from thermal images).

In some example embodiments, the structure of each layer is predefined. For example, a convolution layer may contain small convolution kernels and their respective convolution parameters, and a summation layer may calculate the sum, or the weighted sum, of two pixels of the input image. Training assists in defining the weight coefficients for the summation. The example in FIG. 5 illustrates the CNN technique, where convolutions at each layer 506-511 represent smaller or more dense comparisons and evolutions. An RNN technique may use a similar setup, but with feedback from a previous layer (e.g., layer 507 feeds back to layer 506, and at a next step, layer 506 uses the feedback as an input) or from a previous time (e.g., layer 507 uses a previous output as an input at a next step).

The DNN of FIG. 5 may be trained using an x-ray image, a 3D bone model, and a surface image of patient anatomy as inputs, with outputs labeled (e.g., manually by a surgeon) with a final 3D model of patient anatomy. The 3D model output from the DNN may include one or more locations where the 3D bone model is connected to the surface image. A location may represent a feature extracted from the x-ray for skin of a patient, with a relative location to the 3D bone model determined from the x-ray. The location may connect the 3D bone model to a corresponding location of soft tissue or other patient anatomy that represents a feature extracted from the thermal image stitched surface image. This way, the DNN aligns the 3D bone model to the surface image, such as at a plurality of locations, to create the final 3D model.

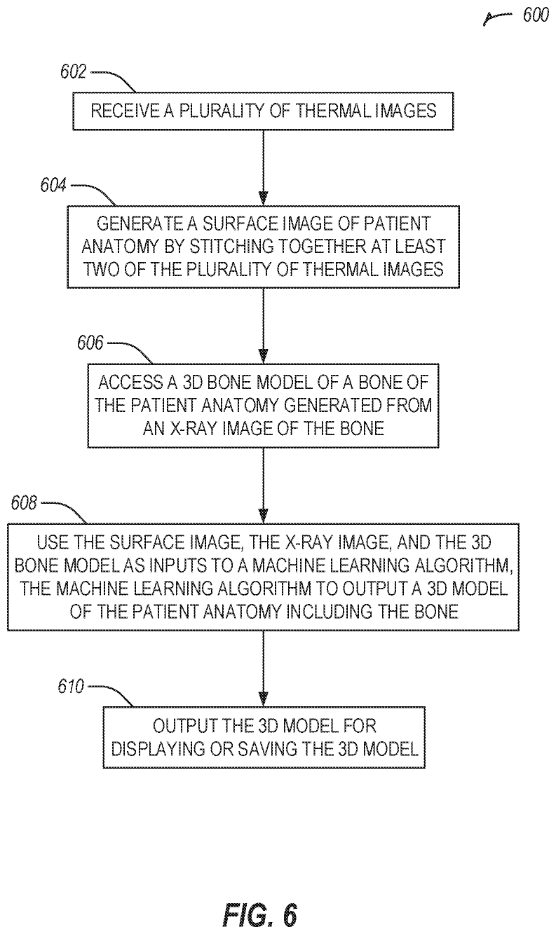

FIG. 6 illustrates a flowchart illustrating a technique 600 for generating a 3D model using thermal imaging and an x-ray in accordance with some embodiments.

The technique 600 includes an operation 602 to receive a plurality of thermal images.

The technique 600 includes an operation 604 to generate a surface image of patient anatomy by stitching together at least two of the plurality of thermal images. Operation 604 may include stitching together at least four thermal images, including thermal images captured from an anterior, a posterior, a medial, and a lateral position.

The technique 600 includes an operation 606 to access a 3D bone model of a bone of the patient anatomy generated from an x-ray image of the bone. The 3D bone model may be generated from a diagnostic x-ray of the bone (e.g., from the x-ray image). Operation 606 may include using two or more x-ray images, such as two images obtained at orthogonal angles, or six x-ray images (e.g., two for hip area, two for knee area, and two for ankle area to fully capture femur and tibia bones) Operation 606 may include accessing 3D bone models of a femur, a tibia, and a fibula, and wherein the 3D bone models are used as inputs to the machine learning algorithm.

The technique 600 includes an operation 608 to use the surface image, the x-ray image (e.g., one of two or more x-rays obtained), or the 3D bone model as inputs to a machine learning algorithm, the machine learning algorithm to output a 3D model of the patient anatomy including, for example, the bone, soft tissue, or the like. The machine learning algorithm may use an outline of patient anatomy other than the bone as a feature (e.g., an outline of the patient's skin from the x-ray). The machine learning algorithm may be a deep neural network, such as a convolutional neural network (CNN) or a recurrent neural network (RNN). The CNN or RNN may be used to match features of the x-ray image (e.g., outline of skin) to features of the surface image such that the 3D bone model may be aligned with the surface image.

The technique 600 includes an operation 610 to output the 3D model for displaying or saving the 3D model. The 3D model may include a combination of the 3D bone model and the surface image. In an example, outputting the 3D model may include generating a preoperative plan for a surgical procedure using the 3D model.

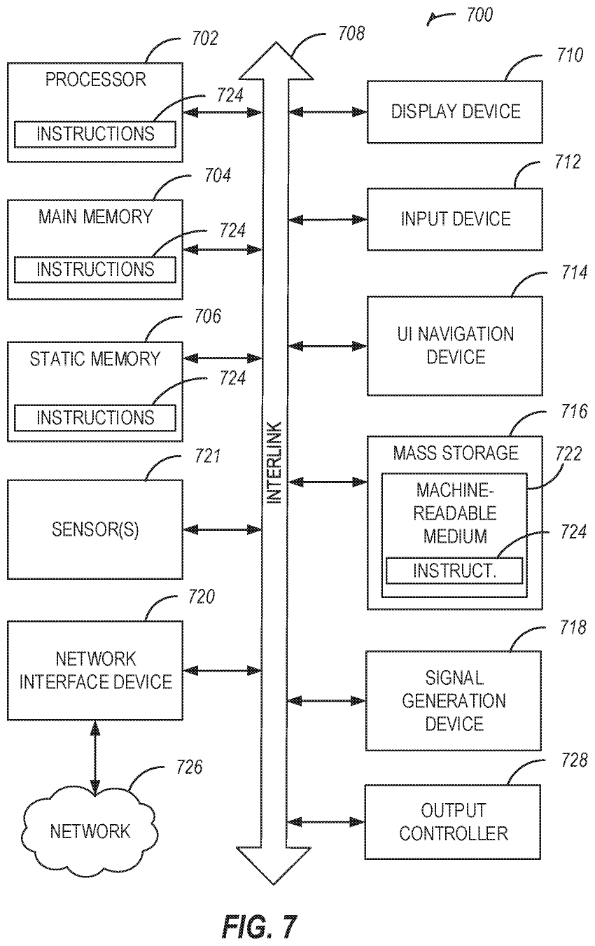

FIG. 7 illustrates a block diagram of an example machine 700 upon which any one or more of the techniques discussed herein may perform in accordance with some embodiments. In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 700 may include a hardware processor 702 (e.g., a central processing unit (CPU), a graphics processing unit (GPU), a hardware processor core, or any combination thereof), a main memory 704 and a static memory 706, some or all of which may communicate with each other via an interlink (e.g., bus) 708. The machine 700 may further include a display unit 710, an alphanumeric input device 712 (e.g., a keyboard), and a user interface (UI) navigation device 714 (e.g., a mouse). In an example, the display unit 710, input device 712 and UI navigation device 714 may be a touch screen display. The machine 700 may additionally include a storage device (e.g., drive unit) 716, a signal generation device 718 (e.g., a speaker), a network interface device 720, and one or more sensors 721, such as a global positioning system (GPS) sensor, compass, accelerometer, or other sensor. The machine 700 may include an output controller 728, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 716 may include a machine readable medium 722 on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the storage device 716 may constitute machine readable media.

While the machine readable medium 722 is illustrated as a single medium, the term “machine readable medium” may include a single medium or multiple media (e.g., a centralized or distributed database, and/or associated caches and servers) configured to store the one or more instructions 724. The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine readable medium examples may include solid-state memories, and optical and magnetic media.

The instructions 724 may further be transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software.

Each of these non-limiting examples may stand on its own, or may be combined in various permutations or combinations with one or more of the other examples.

Example 1 is a method for generating a soft tissue and bone model of a patient, the method comprising: receiving a plurality of thermal images; generating a thermal map of patient anatomy by stitching together at least two of the plurality of thermal images; accessing a 3D bone model of a bone of the patient anatomy generated from an x-ray image (or two x-ray images) of the bone; using the thermal map, the x-ray image, and the 3D bone model as inputs to a machine learning algorithm, the machine learning algorithm to output a 3D model of the patient anatomy including the bone and soft tissue of the patient anatomy; and outputting the 3D model for display.

In Example 2, the subject matter of Example 1 includes, wherein generating the thermal map includes stitching together at least four thermal images, including thermal images captured from an anterior, a posterior, a medial, and a lateral position.

In Example 3, the subject matter of Examples 1-2 includes, generating the 3D bone model from a diagnostic x-ray.

In Example 4, the subject matter of Examples 1-3 includes, wherein accessing the 3D bone model includes accessing a 3D bone model including a distal portion of a femur and a proximal portion of a tibia, and wherein the 3D bone models are used as inputs to the machine learning algorithm.

In Example 5, the subject matter of Examples 1-4 includes, wherein the machine learning algorithm uses an outline of patient anatomy other than the bone as a feature.

In Example 6, the subject matter of Examples 1-5 includes, wherein the 3D model includes a combination of the 3D bone model and the thermal map.

In Example 7, the subject matter of Examples 1-6 includes, wherein the machine learning algorithm is a convolutional neural network (CNN) or a recurrent neural network (RNN).

In Example 8, the subject matter of Example 7 includes, wherein layers of the CNN or the RNN are used to match features of the x-ray image to features of the thermal map.

In Example 9, the subject matter of Examples 1-8 includes, wherein outputting the 3D model includes generating a preoperative plan for a surgical procedure using the 3D model.

Example 10 is a machine-readable medium including instructions for generating a soft tissue and bone model of a patient, the instructions causing a processor to: receive a plurality of thermal images; generate a thermal map of patient anatomy by stitching together at least two of the plurality of thermal images; access a 3D bone model of a bone of the patient anatomy generated from an x-ray image (or two x-ray images) of the bone; use the thermal map, the x-ray image, and the 3D bone model as inputs to a machine learning algorithm, the machine learning algorithm to output a 3D model of the patient anatomy including the bone and soft tissue of the patient anatomy; and output the 3D model for display.

In Example 11, the subject matter of Example 10 includes, wherein to generate the thermal map, the instructions further cause the processor to stitch together at least four thermal images, including thermal images captured from an anterior, a posterior, a medial, and a lateral position.

In Example 12, the subject matter of Examples 10-11 includes, wherein the instructions further cause the processor to generate the 3D bone model from a diagnostic x-ray.

In Example 13, the subject matter of Examples 10-12 includes, wherein to access the 3D bone model, the instructions further cause the processor to access a 3D bone model including a distal portion of a femur and a proximal portion of a tibia, and wherein the 3D bone models are used as inputs to the machine learning algorithm.

In Example 14, the subject matter of Examples 10-13 includes, wherein the machine learning algorithm uses an outline of patient anatomy other than the bone as a feature.

In Example 15, the subject matter of Examples 10-14 includes, wherein the 3D model includes a combination of the 3D bone model and the thermal map.

In Example 16, the subject matter of Examples 10-15 includes, wherein the machine learning algorithm is a convolutional neural network (CNN) or a recurrent neural network (RNN).

In Example 17, the subject matter of Example 16 includes, wherein layers of the CNN or the RNN are used to match features of the x-ray image to features of the thermal map.

In Example 18, the subject matter of Examples 10-17 includes, wherein to output the 3D model, the instructions further cause the processor to generate a preoperative plan for a surgical procedure using the 3D model.

Example 19 is a system for generating a soft tissue and bone model of a patient, the system comprising: a thermal image camera to generate a plurality of thermal images; and a processor coupled to memory, the processor to perform operations comprising: receive an x-ray image (or two x-ray images) of a bone of a patient; generating a thermal map of patient anatomy by stitching together at least two of the plurality of thermal images; generating a 3D bone model of the bone from the x-ray image of the bone; using the thermal map, the x-ray image, and the 3D bone model as inputs to a machine learning algorithm, the machine learning algorithm to output a 3D model of the patient anatomy including the bone and soft tissue of the patient anatomy; and outputting the 3D model for display.

In Example 20, the subject matter of Example 19 includes, wherein generating the thermal map furthers includes stitching together at least four thermal images, including thermal images captured from an anterior, a posterior, a medial, and a lateral position.

In Example 21, the subject matter of Examples 19-20 includes, wherein accessing the 3D bone model includes accessing a 3D bone model including a distal portion of a femur and a proximal portion of a tibia, and wherein the 3D bone models are used as inputs to the machine learning algorithm.

In Example 22, the subject matter of Examples 19-21 includes, wherein the machine learning algorithm uses an outline of patient anatomy other than the bone as a feature.

In Example 23, the subject matter of Examples 19-22 includes, wherein the 3D model includes a combination of the 3D bone model and the thermal map.

In Example 24, the subject matter of Examples 19-23 includes, wherein the machine learning algorithm is a convolutional neural network (CNN) or a recurrent neural network (RNN).

In Example 25, the subject matter of Example 24 includes, wherein layers of the CNN or the RNN are used to match features of the x-ray image to features of the thermal map.

In Example 26, the subject matter of Examples 19-25 includes, wherein outputting the 3D model includes generating a preoperative plan for a surgical procedure using the 3D model.

Example 27 is at least one machine-readable medium including instructions that, when executed by processing circuitry, cause the processing circuitry to perform operations to implement of any of Examples 1-26.

Example 28 is an apparatus comprising means to implement of any of Examples 1-26.

Example 29 is a system to implement of any of Examples 1-26.

Example 30 is a method to implement of any of Examples 1-26.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A method for generating a soft tissue and bone model of a patient, the method comprising:
receiving a plurality of thermal images;
generating a thermal map of patient anatomy by stitching together at least two of the plurality of thermal images;
accessing a 3D bone model of a bone of the patient anatomy generated from two x-ray images of the bone;
using the thermal map, at least one of the two x-ray images, and the 3D bone model as inputs to a machine learning algorithm, the machine learning algorithm to output a 3D model of the patient anatomy including the bone and soft tissue of the patient anatomy; and
outputting the 3D model for display.

2. The method of claim 1, wherein generating the thermal map includes stitching together at least four thermal images, including thermal images captured from an anterior, a posterior, a medial, and a lateral position.

3. The method of claim 1, further comprising generating the 3D bone model from a diagnostic x-ray.

4. The method of claim 1, wherein accessing the 3D bone model includes accessing a 3D bone model including a distal portion of a femur and a proximal portion of a tibia, and wherein the 3D bone models are used as inputs to the machine learning algorithm.

5. The method of claim 1, wherein the machine learning algorithm uses an outline of patient anatomy other than the bone as a feature.

6. The method of claim 1, wherein the 3D model includes a combination of the 3D bone model and the thermal map.

7. The method of claim 1, wherein the machine learning algorithm is a convolutional neural network (CNN) or a recurrent neural network (RNN).

8. The method of claim 7, wherein layers of the CNN or the RNN are used to match features of the at least one of the two x-ray images to features of the thermal map.

9. The method of claim 1, wherein outputting the 3D model includes generating a preoperative plan for a surgical procedure using the 3D model.

10. A machine-readable medium including instructions for generating a soft tissue and bone model of a patient, the instructions causing a processor to:
receive a plurality of thermal images;
generate a thermal map of patient anatomy by stitching together at least two of the plurality of thermal images;
access a 3D bone model of a bone of the patient anatomy generated from two x-ray images of the bone;
use the thermal map, at least one of the two x-ray images, and the 3D bone model as inputs to a machine learning algorithm, the machine learning algorithm to output a 3D model of the patient anatomy including the bone and soft tissue of the patient anatomy; and
output the 3D model for display.

11. The machine-readable medium of claim 10, wherein to generate the thermal map, the instructions further cause the processor to stitch together at least four thermal images, including thermal images captured from an anterior, a posterior, a medial, and a lateral position.

12. The machine-readable medium of claim 10, wherein the instructions further cause the processor to generate the 3D bone model from a diagnostic x-ray.

13. The machine-readable medium of claim 10, wherein to access the 3D bone model, the instructions further cause the processor to access a 3D bone model including a distal portion of a femur and a proximal portion of a tibia, and wherein the 3D bone models are used as inputs to the machine learning algorithm.

14. The machine-readable medium of claim 10, wherein the machine learning algorithm uses an outline of patient anatomy other than the bone as a feature.

15. The machine-readable medium of claim 10, wherein the 3D model includes a combination of the 3D bone model and the thermal map.

16. The machine-readable medium of claim 10, wherein the machine learning algorithm is a convolutional neural network (CNN) or a recurrent neural network (RNN).

17. The machine-readable medium of claim 16, wherein layers of the CNN or the RNN are used to match features of the at least one of the two x-ray images to features of the thermal map.

18. The machine-readable medium of claim 10, wherein to output the 3D model, the instructions further cause the processor to generate a preoperative plan for a surgical procedure using the 3D model.

19. A system for generating a soft tissue and bone model of a patient, the system comprising:
a thermal image camera to generate a plurality of thermal images; and
a processor coupled to memory, the processor to perform operations comprising:
receive two x-ray images of a bone of a patient;
generating a thermal map of patient anatomy by stitching together at least two of the plurality of thermal images;
generating a 3D bone model of the bone from the two x-ray image of the bone;
using the thermal map, at least one of the two x-ray images, and the 3D bone model as inputs to a machine learning algorithm, the machine learning algorithm to output a 3D model of the patient anatomy including the bone and soft tissue of the patient anatomy; and
outputting the 3D model for display.

20. The system of claim 19, wherein generating the thermal map furthers includes stitching together at least four thermal images, including thermal images captured from an anterior, a posterior, a medial, and a lateral position.
